(19) United States
(12) Patent Application Publication
(10) Pub. No.: US 2025/0151726 A1
(43) Pub. Date: May 15, 2025

(54) DISINFECTION PRODUCT BASED ON CITRIC ACID AND NATURAL NAPHTHOQUINONE COMPOUND

(71) Applicant: SHANGHAI JIAO TONG UNIVERSITY, Shanghai (CN)

(72) Inventors: Jinping JIA, Shanghai (CN); Zhizhuo LIU, Shanghai (CN); Jiahua CUI, Shanghai (CN)

(73) Assignee: SHANGHAI JIAO TONG UNIVERSITY, Shanghai (CN)

(21) Appl. No.: 18/832,935

(22) PCT Filed: Mar. 4, 2022
(86) PCT No.: PCT/CN2022/079238

§ 371 Date: Jul. 25, 2024

(30) Foreign Application Priority Data
Jan. 26, 2022 (CN) 202210094119.4

(51) Int. Cl. A01N 37/36; A01N 25/04; A01N 35/06; A01P 1/00

(52) U.S. Cl. CPC A01N 37/36; A01N 25/04; A01N 35/06; A01P 1/00

(57) ABSTRACT

A disinfection product based on citric acid and a natural naphthoquinone compound, and a preparation method thereof are provided. The disinfectant includes, in weight percentage, 15-25% of citric acid, 0.01-0.1% of a natural naphthoquinone compound, a certain amount of ethanol, and a certain amount of deionized water containing an alkali metal salt and an alkaline-earth metal salt. When in use, the disinfectant can be sprayed on the surface of an object or the diluted disinfectant can be used for cleaning an object to be disinfected. The disinfection product features high disinfection and sterilization efficacy, low irritation, and low corrosiveness, and is free from peculiar smell and environmental toxicity in disinfection residues. The disinfectant is suitable for the disinfection of cold chain and the disinfectant of surfaces of vegetable and food.

### CROSS REFERENCE TO THE RELATED APPLICATIONS

This application is the national phase entry of International Application No. PCT/CN2022/079238, filed on Mar. 4, 2022, which is based upon and claims priority to Chinese Patent Application No. 202210094119.4, filed on Jan. 26, 2022, the entire contents of which are incorporated herein by reference.

### TECHNICAL FIELD

The present invention relates to the research field of disinfectant and bactericidal preparations as well as disinfection gels for military and civilian applications, specifically to a disinfection product based on citric acid and a natural naphthoquinone compound, and a preparation method thereof, and particularly to an environmentally-friendly disinfection solution and disinfection gel based on citric acid and a natural naphthoquinone, and preparation methods thereof.

### BACKGROUND

People have been plagued by epidemics from the spread of germs or viral infections since the birth of humanity. An outbreak caused by severe acute respiratory syndrome coronavirus 2 (SARS-COV-2) spread worldwide in early 2020 and has become a public health event of common concern around the world. In previous studies, we detected the SARS-COV-2 or its nucleic acid residues on solid surfaces, and in feces, domestic sewage, and even imported frozen foods. Some studies have shown that human coronaviruses can survive on a dry surface for up to nine days (Kampf, G., Todt, D., Pfaender, S. & Steinmann, E. Persistence of coronaviruses on inanimate surfaces and their inactivation with biocidal agents. J Hosp Infect 104, 246-251, (2020)). Therefore, the disinfectants are employed to kill SARS-COV-2 in public places, which may help prevent further virus spreading.

Chemical disinfectants that have been widely used currently, such as chlorine-containing disinfectant (84 disinfectant), peroxyacetic acid, ethanol, and quaternary ammonium disinfectant, feature certain oxidation, corrosion, and sensitization. Repeatedly spraying disinfectant in a large area during epidemic prevention and control also brings about environmental safety issues and secondary risks, and poses threats to human health. Water is one of the major objects to be disinfected by disinfectants, and chlorine plays a major role in the disinfection of drinking water and wastewater. However, there is evidence that the chlorine produces a variety of disinfection by-products (DBPs) with environmental toxicity, such as trichloromethane and haloacetic acid, after entering the environmental water body. These DBPs pose a potential carcinogenic risk to human bodies. In 2013, the European Academy of Allergy and Clinical Immunology (EAACI) pointed out that the increased risk of work-related asthma (WRA), work-exacerbated asthma (WEA), and rhinitis for dustmen is mainly associated with the exposure to cleaning sprays, bleachers, ammonium hydroxide, disinfectants, and mixed products. According to a follow-up study conducted in the United States between 2009 and 2015, the nurses' exposure to the cleaning products and disinfectants increases their risk of suffering from the asthma by 25% to 38% (Folletti, I., Siracusa, A. & Paolocci, G. Update on asthma and cleaning agents. Curr Opin Allergy Clin Immunol 17, 90-95, (2017)).

Hence, it is imperative to develop a disinfectant with low toxicity but high efficacy for environmental disinfection protection, while effectively controlling the negative effects of disinfectants on human bodies and the environments and coping with the increasing number of multi-drug resistance (MDR) bacteria.

Natural products are secondary metabolites of plants, animals, and microorganisms. Some of the natural products have significant antibacterial and antiviral activities. The natural products are low in toxicity and little in environmental harm. Since ancient times, mugwort has been documented for its function as an insect repellent and an antibacterial agent in China. During the severe acute respiratory syndrome (SARS) outbreak in 2003 and the SARS-COV-2 outbreak in 2020, traditional Chinese medicinal materials with aroma and dryness, such as mugwort, cangzhu, and Angelica dahurica, were reported for hospital disinfection by fumigation. (Zhang Jiale et al. Analysis of the Application of Moxa Leaf Fumigation in air disinfection of COVID-19. Journal of Gannan Medical University, 41, 254-258, (2020).)

### SUMMARY

An object of the present invention is to overcome the above-mentioned deficiencies of the prior art and to provide a disinfection product based on citric acid and a natural naphthoquinone compound, and a preparation method thereof. The disinfection product is a disinfectant or a disinfection gel with environmental friendliness, green and environmental protection, low irritation, and low corrosiveness, which can be used for sterilization and disinfection.

The objective of the present invention may be implemented by the following technical solution:

The present invention provides a disinfection product based on citric acid and a natural naphthoquinone compound, including the following components in parts by weight:
15-25 parts of citric acid; and
0.01-1 part of a naphthoquinone compound.

As an embodiment of the present invention, the naphthoquinone compound includes one or more of juglone, 7-methyljuglone, 7-methyl-5-acetyl juglone, rheum emodin, and alkannin.

As an embodiment of the present invention, the citric acid has a purity greater than 99.5%.

As an embodiment of the present invention, the disinfection product further includes 0.1-1 part of an inorganic salt in parts by weight.

As an embodiment of the present invention, the inorganic salt comprises an alkali metal salt and/or an alkaline-earth metal salt. The inorganic salt is analytically pure grade. The alkali metal salt and the alkaline-earth metal salt can absorb moisture from the air due to the deliquescence effect, which increases the effective action time of the disinfection product in daily use and enhances the long-term inhibitory ability.

As an embodiment of the present invention, the alkali metal salt includes one or more of sodium chloride (NaCl), potassium sulphate, and potassium nitrate; and the alkaline-earth metal salt includes one or more of magnesium chloride, magnesium sulphate, and calcium sulphate.

As an embodiment of the present invention, the disinfection product further includes 1-5 parts of a cosolvent in parts by weight. The cosolvent includes one of ethanol, isopropanol, and dimethylsulfoxide (DMSO).

The present invention further provides a use of a disinfection product based on citric acid and a natural naphthoquinone compound in the preparation of a disinfection solution and a disinfection gel.

The present invention provides a disinfection solution, including, per hundred parts, the following components in parts by weight:
15-25 parts of citric acid;
1-5 parts of a cosolvent;
0.01-1 part of a naphthoquinone compound;
0.1-1 part of an inorganic salt; and
the balance of deionized water.

As an embodiment of the present invention, the cosolvent includes one of ethanol, isopropanol, and DMSO. The naphthoquinone compound is dissolved into the cosolvent and then mixed with other ingredients of the disinfection solution, which makes the mass fraction of the naphthoquinone compound in the disinfection solution be 0.01-1%.

The present invention further provides a preparation method for the disinfection solution, including the following steps:
S1, weighing and adding the citric acid into an appropriate amount of deionized water, and heating to 70-95° C. in a water bath reaction kettle to ensure that the citric acid is fully dissolved in the preparation process;
S2, weighing and adding the inorganic salt into the aqueous citric acid solution prepared in the step S1, and stirring at a stirring speed of 60-100 r/min at 70-95° C. for 25-35 minutes; and
S3, cooling the solution obtained in the step S2 to room temperature, followed by adding a mixed solution formed by dissolving the naphthoquinone compound into the cosolvent, to make the mass fraction of the naphthoquinone compound in the disinfection solution be 0.01-1%, and stirring evenly to obtain the disinfection solution.

The present invention provides a disinfection gel, including, per hundred parts, the following components in parts by weight:
15-25 parts of citric acid;
0.01-0.1 parts of a naphthoquinone compound;
0.1-1 part of an inorganic salt;
1-5 parts of a cosolvent;
1-3 parts of a thickener; and
the balance of deionized water.

As an embodiment of the present invention, the cosolvent includes one of ethanol, isopropanol, and DMSO. The naphthoquinone compound is dissolved into the cosolvent and then mixed with other ingredients of the disinfection solution, which makes the mass fraction of the naphthoquinone compound in the disinfection solution be 0.01-1%.

As an embodiment of the present invention, the thickener is prepared by adding hydroxyethyl cellulose to an ethanol solution containing deionized water, an acetic acid buffer solution, and chlorhexidine acetate. The amount ratio of the hydroxyethyl cellulose to the deionized water to the acetic acid buffer solution to the chlorhexidine acetate is 10 g: 18.5 mL: 1 mL: 0.5 mL. By adding thickening ingredients, the disinfectant becomes gelatinous, and is easy to carry and use.

The present invention further provides a preparation method for the disinfection gel, including the following steps:
A1, weighing and adding the citric acid into an appropriate amount of deionized water, and heating to 70-95° C. in a water bath reaction kettle to ensure that the citric acid is fully dissolved in the preparation process;
A2, weighing and adding the inorganic salt into the aqueous citric acid solution obtained in the step A1, and stirring at a stirring speed of 60-100 r/min at 70-95° C. for 25-35 minutes; and
A3, cooling the solution obtained in the step A2 to room temperature, followed by adding a mixed solution formed by dissolving the naphthoquinone compound into the cosolvent, to make the mass fraction of the naphthoquinone compound in the disinfection solution be 0.01-1%, adding the thickener after stirring evenly, and mixing and thickening to obtain the disinfection gel.

The preparation method according to the present invention can significantly improve the solubility of the salts.

The preparation method according to the present invention is conducive to improving the synergistic effect of the citric acid and the inorganic salt. In the step A2, heating and stirring the citric acid and the inorganic salt can cause a complex reaction, thereby facilitating the increase in the concentration of effective bactericidal ingredients in the disinfectant. The amount of each component in the present invention is optimal, and the amount of the citric acid reaches the maximum solubility thereof. Adding excessive citric acid will generate crystals at room temperature, thus reducing the effect; and adding too little citric acid will decrease the concentration thereof, thus reducing the bactericidal effect. The amount of the naphthoquinone compound also reaches the maximum solubility thereof. Adding excessive naphthoquinone compound will separate out substances in the disinfectant, thus reducing the stability of the disinfectant; and adding too little naphthoquinone compound will decrease the concentration thereof, thus reducing the bactericidal effect. Excessive inorganic salt will lead to a decrease in stability of the disinfectant, and too little inorganic salt is not conducive to the extension of the bactericidal effect of the disinfectant.

The disinfectant solution prepared in the present invention can be sprayed on the object surfaces, and features disinfection and sterilization, little irritation, and small corrosion, and is free from peculiar smell and environmental toxicity in disinfection residues.

Compared with the prior art, the present invention has the following beneficial effects:
1) By adding the citric acid, an ingredient in natural plants, the disinfectant has effects capable of killing viruses and bacteria, and is certainly acidic and green, with low toxicity.
2) By adding the natural naphthoquinone compound, a new antibacterial and antiviral target is provided to improve the efficiency of the disinfectant in killing bacteria and viruses.
3) An environmentally-friendly, green, safe, and efficient novel disinfectant is prepared by using the citric acid, the natural naphthoquinone compound, and the inorganic salt that are low in toxicity and free of pollution.
4) Through the perforation of the citric acid, the natural naphthoquinone compound can enter bacterial cells in the form of free diffusion, thus improving the bactericidal efficiency of the naphthoquinone compound.
5) The citric acid and the naphthoquinone compound have a good synergistic effect and feature low toxicity, so they are the best components for the disinfectant.

### BRIEF DESCRIPTION OF THE DRAWINGS

By reading the detailed description of the non-restrictive examples with reference to the following accompanying drawings, other features, objects, and advantages of the present invention will become more apparent:

FIG. 1 is a schematic diagram of the minimal inhibitory concentration of disinfection solutions prepared in Example 1 and Comparative Examples 1-4.

FIG. 2 is a schematic diagram of inhibitory time in Example 1 and Comparative Example 5.

FIG. 3 is a schematic diagram of the inhibitory effect of a disinfection solution in Example 1.

FIG. 4 is a schematic diagram of the bactericidal effect of the disinfection solution in Example 1.

FIG. 5 is a change chart of heartbeats of zebrafishes treated with the disinfection solution in Example 1 at different concentrations.

FIG. 6 is a change chart of the number of wagging of zebrafishes treated with the disinfection solution in Example 1 at different concentrations.

FIG. 7 is a change chart of weights of mice treated with the disinfection solution in Example 1 at different concentrations.

### DETAILED DESCRIPTION OF THE EMBODIMENTS

The present invention is described in detail below with reference to the accompanying drawings and specific examples. The following examples facilitate those skilled in the art to further appreciate the present invention, but do not limit the present invention in any way. It should be pointed out that a number of adjustments and improvements can be made by those of ordinary in the art, without deviating from the concept of the present invention. All of these belong to the protection scope of the present invention. Experimental methods without specified conditions in the following examples usually follow conventional conditions or conditions recommended by the manufacturers.

The present invention discloses a disinfection solution based on citric acid and a natural naphthoquinone compound, and a preparation method thereof. Those skilled in the art can learn from the text content and appropriately improve process parameters to realize the disinfection solution. It's important to note that all similar replacements and alterations would become apparent to those skilled in the art and be considered to be included in the scope of the present invention.

### Example 1

The example relates to a method for preparing a disinfection solution based on citric acid and a natural naphthoquinone compound, the method including the following steps:
(1) precisely weighing 48 g of citric acid having a purity greater than 99.5%, 0.7 g of analytically pure NaCl, 2 mL of a juglone ethanol solution having a concentration of 100 μM, and 200 mL of purified water for further use;
(2) adding the citric acid and the NaCl to 200 mL of the purified water, and heating to 70-95° C. in a water bath reaction kettle to ensure that the citric acid is fully dissolved in the preparation process;
(3) cooling the solution prepared in the step (2) to room temperature, adding 2 mL of the juglone ethanol solution having the concentration of 100 μM, and stirring evenly to obtain the disinfection solution based on the citric acid and the natural naphthoquinone; and
(4) subpackaging the finally obtained solution into spray bottles.

### Example 2

The example relates to a method for preparing a disinfection gel based on citric acid and a natural naphthoquinone compound.
(1) precisely weighing 40 g of citric acid having a purity greater than 99.5%, 0.3 g of analytically pure potassium sulphate, 2 mL of a rheum emodin ethanol solution having a concentration of 100 μM, and 200 mL of purified water for further use;
(2) adding the citric acid and the potassium sulphate to 200 mL of the purified water, and heating to 70-95° C. in a water bath reaction kettle to ensure that the citric acid is fully dissolved in the preparation process;
(3) cooling the solution prepared in the step (2) to room temperature, adding 2 mL of the rheum emodin ethanol solution having the concentration of 100 μM, and stirring evenly; and
(4) mixing the solution prepared in the step (3) with a thickener having a mass percentage of 3%, with an amount ratio of hydroxyethyl cellulose, deionized water, an acetic acid buffer solution, and chlorhexidine acetate of 10 g: 18.5 mL: 1 mL: 0.5 mL in the thickener, and obtaining the disinfection gel after thickening; and
(5) subpackaging the finally obtained gel into bottles.

### Example 3

The example relates to a method for preparing a disinfection solution based on citric acid and a natural naphthoquinone compound.
(1) precisely weighing 38 g of citric acid having a purity greater than 99.5%, 0.28 g of analytically pure NaCl, 2 mL of a 7-methyljuglone ethanol solution having a concentration of 100 μM, and 198 mL of purified water for further use;
(2) adding the citric acid and the NaCl to 198 mL of the purified water, and heating to 70-95° C. in a water bath reaction kettle to ensure that the citric acid is fully dissolved in the preparation process;
(3) cooling the solution prepared in the step (2) to room temperature, adding 2 mL of the 7-methyljuglone ethanol solution having the concentration of 100 μM, and stirring evenly to obtain the disinfection solution based on the citric acid and the natural naphthoquinone; and
(4) subpackaging the finally obtained solution into spray bottles.

### Example 4

The example relates to a method for preparing a disinfection solution based on citric acid and a natural naphthoquinone compound.
(1) precisely weighing 64 g of citric acid having a purity greater than 99.5%, 0.28 g of analytically pure NaCl, 0.1 mL of a 7-methyl-5-acetyl juglone ethanol solution having a concentration of 100 μM, and 200 mL of purified water for further use;
(2) adding the citric acid and the NaCl to 200 mL of the purified water, and heating to 70-95° C. in a water bath reaction kettle to ensure that the citric acid is fully dissolved in the preparation process;
(3) cooling the solution prepared in the step (2) to room temperature, adding 0.1 mL of the 7-methyl-5-acetyl juglone ethanol solution having the concentration of 100 μM, and stirring evenly to obtain the disinfection solution based on the citric acid and the natural naphthoquinone; and
(4) subpackaging the finally obtained solution into spray bottles.

### Example 5

The example relates to a method for preparing a disinfection solution based on citric acid and a natural naphthoquinone compound.
(1) precisely weighing 38 g of citric acid having a purity greater than 99.5%, 0.54 g of analytically pure NaCl, 2 mL of an alkannin ethanol solution having a concentration of 100 μM, and 198 mL of purified water for further use;
(2) adding the citric acid and the NaCl to 198 mL of the purified water, and heating to 70-95° C. in a water bath reaction kettle to ensure that the citric acid is fully dissolved in the preparation process;
(3) cooling the solution prepared in the step (2) to room temperature, adding 2 mL of the alkannin ethanol solution having the concentration of 100 μM, and stirring evenly to obtain the disinfection solution based on the citric acid and the natural naphthoquinone; and
(4) subpackaging the finally obtained solution into spray bottles.

### Comparative Example 1

The comparative example relates to a method for preparing a disinfection solution based on citric acid and a natural naphthoquinone compound. The method is basically the same as that in Example 1, except that the amount of citric acid is 20 g and the amount of purified water is 198 mL.

The minimal inhibitory concentration (MIC) of the disinfection solution is determined by a 96-well plate method which is the same as that in Example 1. The MIC of the disinfection solution is 100-fold dilution in Comparative Example 1, with a decrease in disinfection effect compared with an original disinfection solution.

### Comparative Example 2

The comparative example relates to a method for preparing a disinfection solution based on citric acid and a natural naphthoquinone compound. The method is basically the same as that in Example 1, except that no juglone is contained.

The MIC of the disinfection solution is determined by a 96-well plate method which is the same as that in Example 1. The MIC of the disinfection solution is 150-fold dilution in Comparative Example 2, with a decrease in disinfection effect compared with an original disinfection solution.

### Comparative Example 3

The comparative example relates to a method for preparing a disinfection solution based on citric acid and a natural naphthoquinone compound. The method is basically the same as that in Example 1, except that the juglone is replaced by 1,4-naphthoquinone.

The MIC of the disinfection solution is determined by a 96-well plate method which is the same as that in Example 1. The MIC of the disinfection solution is 100-fold dilution in Comparative Example 3, with a decrease in disinfection effect compared with an original disinfection solution, and the juglone is more economical.

### Comparative Example 4

The comparative example relates to a method for preparing a disinfection solution based on citric acid and a natural naphthoquinone compound. The method is basically the same as that in Example 1, except that the citric acid is replaced with lactic acid.

The MIC of the disinfection solution is determined by a 96-well plate method which is the same as that in Example 1. The MIC of the disinfection solution is 10-fold dilution in Comparative Example 4, with a decrease in disinfection effect compared with an original disinfection solution.

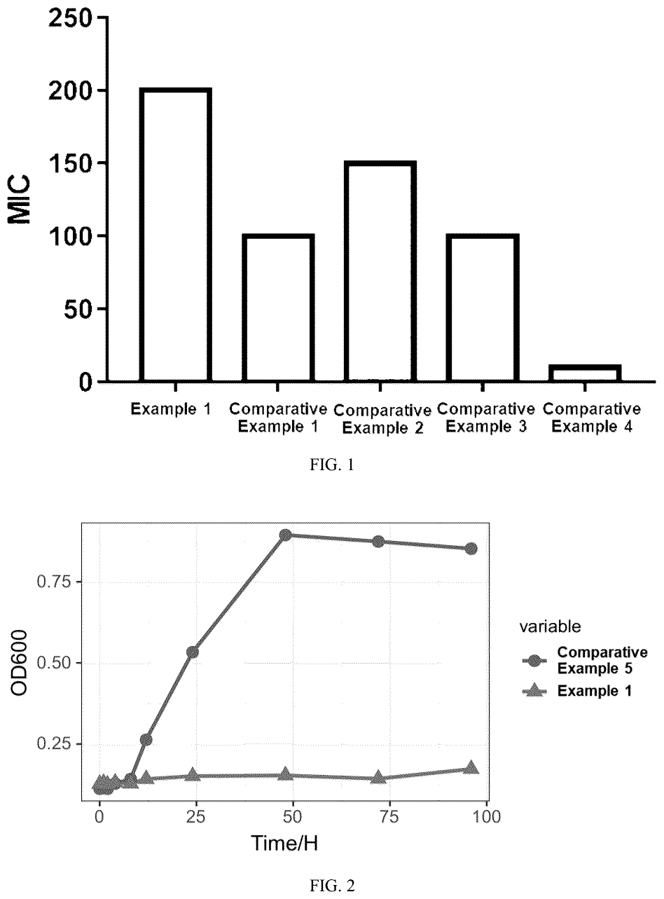

As shown in FIG. 1, the disinfection and inhibitory effects are apparently reduced in Comparative Examples 1˜4 compared with Example 1. The bactericidal effect of organic acids, such as lactic acid, is not as good as that of the citric acid, and the complexation effect of other acids and salts is poor.

### Comparative Example 5

The comparative example relates to a method for preparing a disinfection solution based on citric acid and a natural naphthoquinone compound. The method is basically the same as that in Example 1, except that the inorganic salt is not added.

The MIC of the disinfection solution is determined by a 96-well plate method which is the same as that in Example 1. The MIC of the disinfection solution is 10-fold dilution in Comparative Example 5, with a decrease in disinfection effect compared with an original disinfection solution. As shown in FIG. 2, the effective inhibitory time is apparently shortened.

### Effect Verification I: Inhibition Kinetics Experiment on Disinfection Solution Prepared in Example 1.

This experiment relates to the method and result of the inhibition kinetics experiment on the disinfection solution based on the citric acid and the natural naphthoquinone compound in Example 1. During the determination, bacteria are re-suspended in a Luria-Bertani (LB) medium system, and the disinfection solution is also diluted with the LB medium to give bacteria sufficient growth conditions, thereby simulating the inhibition of bacterial growth by the disinfection solution in an environment rich in nutrients.

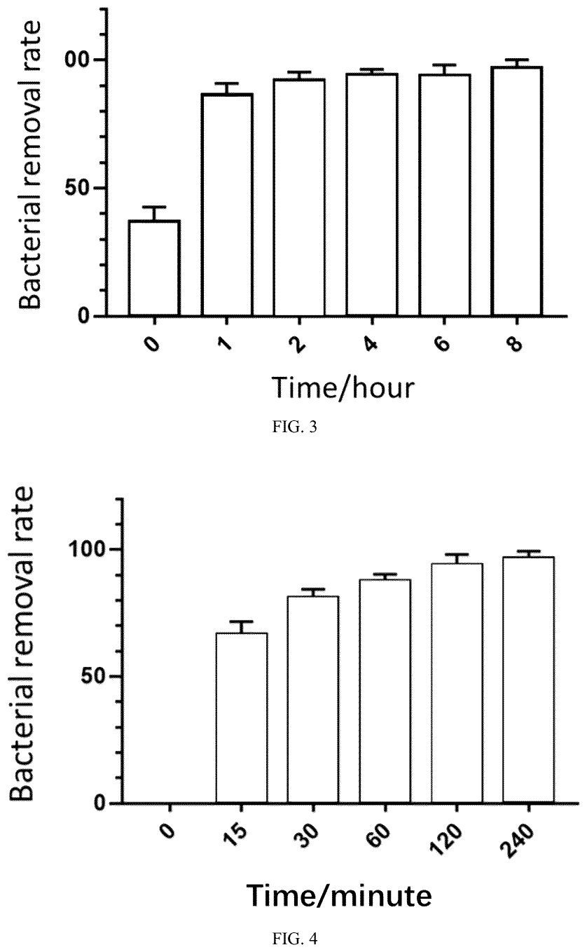

According to the standards of the Clinical & Laboratory Standards Institute (CLSI) of the United States, the inhibitory effect of the disinfection solution was evaluated through the improved microplate method. The specific experimental steps are as follows:
(1) A Staphylococcus aureus suspension in a logarithmic growth phase upon overnight cultivation was taken, and an OD600 value thereof was determined with a microplate reader; and if the OD value is between 0.5 and 0.8, the Staphylococcus aureus is considered to be in the logarithmic growth phase.
(2) The suspension was diluted 100 folds using a high-temperature sterilized LB medium under an aseptic condition, followed by adding 100 μL of the diluted suspension to each microwell in a 96-well plate using a multichannel pipette.
(3) The disinfection solution that was diluted 50 folds using a high-temperature sterilized LB medium was added at a volume of 100 μM to each of 18 parallel microwells containing the suspension.
(4) The 96-well plate was placed in a thermostatic incubator for cultivation at 37° C., followed by pipetting the suspension from three microwells at 0 h, 1 h, 2 h, 4 h, and 8 h respectively, and then transferring the suspension in the microwells at different concentration gradients to different 1.5 mL polyethylene (PE) tubes using the pipette, and placing the PE tubes in ice immediately.
(5) The suspension was centrifugated at 10,000 r/min for 10 min using a high-speed centrifuge and re-suspend with a 0.85% NaCl solution, with three repeats for this process.
(6) Bacteria were stained with a LIVE/DEAD stain in the dark and incubated for 15 min at room temperature.
(7) Bacterial flora was determined using a flow cytometry to perform bacteria counting and characterize the inhibitory activity of the disinfection solution based on the numbers of live cells and dead cells at different concentration gradients. Experiment results are shown in FIG. 3.

According to the experiment results, it can be observed that the longer the treatment time by the novel disinfection solution according to the present invention, the higher the removal rate of Staphylococcus aureus. Despite achieving a removal rate of less than 40% at 1 h, the removal rate of Staphylococcus aureus exceeds 90% after two hours of treatment. Moreover, the inhibitory efficacy continues to escalate within 8 h, with an inhibitory rate of 98.3% at 8 h. This result demonstrates that the novel disinfection solution according to the present invention has a stable ability to continuously inhibit bacterial growth.

### Effect Verification II: Disinfection Kinetics Experiment on Disinfection Solution Prepared in Example 1.

This experiment relates to the method and result of the disinfection kinetics experiment on the disinfection solution based on the citric acid and the natural naphthoquinone compound in Example 1. During the determination, bacteria are re-suspended in a 0.85% NaCl solution, and the disinfection solution is also diluted with the 0.85% NaCl solution, so that energy substances such as organic matter required for bacterial survival are not given to avoid bacterial reproduction. Under the above conditions, the effect of disinfection solution in killing bacteria directly in an environment lacking nutrient supply is determined.

According to the standards of CLSI of the United States, the disinfection effect of the disinfection solution was evaluated through the improved microplate method. The specific experimental steps are as follows:
(1) A Staphylococcus aureus suspension in a logarithmic growth phase upon overnight cultivation was taken, and an OD600 value thereof was determined with a microplate reader; and if the OD value is between 0.5 and 0.8, the Staphylococcus aureus is considered to be in the logarithmic growth phase.
(2) The suspension was diluted 100 folds using a high-temperature sterilized 0.85% NaCl solution under an aseptic condition, followed by adding 100 μL of the diluted suspension to each microwell in a 96-well plate using a multichannel pipette.
(3) The disinfection solution that was diluted 50 folds using the high-temperature sterilized 0.85% NaCl solution was added at a volume of 100 μL to each of 18 parallel microwells containing the suspension.
(4) The 96-well plate was placed in a thermostatic incubator for cultivation at 37° C., followed by pipetting the suspension from three microwells at 0 h, 15 min, 30 min, 1 h, 2 h, and 4 h respectively, and then transferring the suspension in the microwells at different concentration gradients to different 1.5 mL PE tubes using the pipette, and placing the PE tubes in ice immediately.
(5) The suspension was centrifugated at 10,000 r/min for 10 min using a high-speed centrifuge and re-suspend with the 0.85% NaCl solution, with three repeats for this process.
(6) Bacteria were stained with a LIVE/DEAD stain in the dark and incubated for 15 min at room temperature.
(7) Bacterial flora was determined using a flow cytometry to perform bacterial counting and characterize the inhibitory activity of the disinfection solution based on the numbers of live cells and dead cells at different concentration gradients. Experiment results are shown in FIG. 4.

According to the experiment results, we can observe that the longer the treatment time by the novel disinfection solution according to the present invention, the higher the removal rate of Staphylococcus aureus. The removal rate of Staphylococcus aureus reaches nearly 70% at 15 min and 96.7% at 240 min, and bacteria detected in the flow cytometry have severely damaged cell membranes, with the viability and pathogenicity greatly reduced. This result demonstrates that the novel disinfection solution according to the present invention has a strong ability to kill bacteria within short time.

### Effect Verification III: Skin Irritation Experiment on Disinfection Solution Prepared in Example 1.

This experiment relates to the skin irritation experiment on the disinfection solution based on the citric acid and the natural naphthoquinone compound in Example 1.

With reference to the Toxicological Procedures and Methods of Safety Evaluation for Disinfectant (GB/T 38496-2020), the safety of the disinfection solution based on the citric acid and the natural naphthoquinone compound was evaluated using a New Zealand rabbit in this experiment.

The specific experiment steps are as follows:
(1) An adult New Zealand rabbit was taken, and the hair on both sides of the spine on the back of the New Zealand rabbit was removed using scissors 24 h before the start of the experiment.
(2) 1 mL of subject was infiltrated on 2 to 4 layers of gauze in a size of 2.5 cm*2.5 cm, followed by applying the gauze to a fur surface on one side, and then securing with a non-irritating plastic film and an adhesive tape. A group of blank control and six experimental groups including 20-fold diluted disinfection solution, 10-fold diluted disinfection solution, 5-fold diluted disinfection solution, 20-fold diluted 84 disinfectant, 10-fold diluted 84 disinfectant, and 5-fold diluted 84 disinfectant were set.
(3) After application for 4 h, the residual subject was removed with a non-irritating solvent.
(4) Local skin reactions were observed at 1 h, 24 h, and 48 h after removal of the subject and scored according to the skin irritation rating scale in the Toxicological Procedures and Methods of Safety Evaluation for Disinfectant (GB/T 38496-2020). The specific scoring criteria are as follows:

| | | Skin irritation |
|---|---|---|
| | Skin irritation reaction | reaction scoring |
| Erythema formation | None | 0 |
| | Faintly visible | 1 |
| | Obvious | 2 |
| | Severe | 3 |
| | Purplish erythema | 4 |
| | with eschar | |
| Edematization | None | 0 |
| | Faintly visible | 1 |
| | Elevated skin with | 2 |
| | distinct borders | |
| | Swelling raised | 3 |
| | approximately 1 mm | |
| | Swelling raised over 1 | 4 |
| | mm | |

The results of a complete skin irritation experiment are as follows:

TABLE 1
Scores of different subjects in complete skin irritation experiment

| | 1 h | | 24 h | | 48 h | |
|---|---|---|---|---|---|---|
| | Ery- | | Ery- | | Ery- | |
| Subject | thema | Edema | thema | Edema | thema | Edema |
| 20% of 84 | 3 | 2 | 3 | 2 | 2 | 1 |
| disinfectant | | | | | | |
| 10% of 84 | 2 | 2 | 2 | 1 | 1 | 0 |
| disinfectant | | | | | | |
| 5% of 84 | 1 | 1 | 2 | 1 | 0 | 0 |
| disinfectant | | | | | | |
| 20% of | 1 | 1 | 1 | 1 | 0 | 0 |
| disinfection | | | | | | |
| solution | | | | | | |
| 10% of | 0 | 0 | 0 | 0 | 0 | 0 |
| disinfection | | | | | | |
| solution | | | | | | |
| 5% of | 0 | 0 | 0 | 0 | 0 | 0 |
| disinfection | | | | | | |
| solution | | | | | | |

According to the experiment results, we can observe that both erythema and edema are obviously formed at 1 h after treatment with 20% of 84 disinfectant, especially the erythema is more serious and lasts until 24 h, and slightly weakens only after 48 h. After applying the novel disinfection solution with the same concentration according to the present invention, the skin only has faintly visible erythema and edema, and completely recovers after 48 h. The skin of the New Zealand rabbit treated with 10% of 84 disinfectant and 5% of 84 disinfectant has erythema and edema at varying degrees, but no erythema or edema appears on that treated with the disinfection solution with the same concentration. This result demonstrates that the novel disinfection solution according to the present invention has very little irritation to the rabbit skin at effective bactericidal concentrations, much lower than the 84 disinfectant, showing that the novel disinfection solution according to the present invention is safer.

### Effect Verification IV: Ecotoxicity Evaluation Experiment on Disinfection Solution Prepared in Example 1.

This experiment relates to the ecotoxicity evaluation of the disinfection solution based on the citric acid and the natural naphthoquinone compound in Example 1.

The environmental toxicity evaluation of a zebrafish model is carried out in this experiment, including an acute toxicity experiment and a behavioral experiment. The specific experiment steps are as follows:

### I. Acute Toxicity Experiment

(1) The disinfection solution and an 84 disinfectant were diluted with an Omron's Drying (OD) solution to make final concentrations reach 20%, 10%, 5%, and 1%, respectively (calculation of volumetric ratios based on concentrations).

(2) 20 zebrafish embryos and 20 juvenile zebrafishes were added to the OD solution containing the disinfection solution at different concentrations, followed by observing the survival of zebrafish embryos and juvenile zebrafishes at 5 min, 30 min, and 90 min, respectively.

The acute toxicity experiment results of the zebrafish are as follows:

TABLE 2
Acute Toxicity Experiment Results of Zebrafish

| | 5 min | 30 min | 90 min |
|---|---|---|---|
| Embryos treated with | 0% | 0% | 0% |
| 20% of 84 disinfectant | | | |
| Embryos treated with | 100% | 0% | 0% |
| 10% of 84 disinfectant | | | |
| Embryos treated with 5% | 100% | 100% | 100% |
| of 84 disinfectant | | | |
| Embryos treated with 1% | 100% | 100% | 100% |
| of 84 disinfectant | | | |
| Embryos treated with | 100% | 100% | 100% |
| 20% disinfection | | | |
| solution | | | |
| Embryos treated with | 100% | 100% | 100% |
| 10% disinfection | | | |
| solution | | | |
| Embryos treated with 5% | 100% | 100% | 100% |
| disinfection solution | | | |
| Embryos treated with 1% | 100% | 100% | 100% |
| disinfection solution | | | |
| Juvenile fishes treated | 0% | 0% | 0% |
| with 20% of 84 | | | |
| disinfectant | | | |
| Juvenile fishes treated | 0% | 0% | 0% |
| with 10% of 84 | | | |
| disinfectant | | | |
| Juvenile fishes treated | 0% | 0% | 0% |
| with 5% of 84 | | | |
| disinfectant | | | |
| Juvenile fishes treated | 100% | 70% | 15% |
| with 1% 84 disinfectant | | | |
| Juvenile fishes treated | 100% | 45% | 5% |
| with 20% disinfection | | | |
| solution | | | |
| Juvenile fishes treated | 100% | 100% | 35% |
| with 10% disinfection | | | |
| solution | | | |
| Juvenile fishes treated | 100% | 100% | 95% |
| with 5% disinfection | | | |
| solution | | | |
| Juvenile fishes treated | 100% | 100% | 100% |
| with 1% disinfection | | | |
| solution | | | |

According to the results shown in Table 2, it can be seen that, for the zebrafish embryos or the juvenile zebrafishes, the lethality of the disinfection solution according to the present invention is lower than that of the 84 disinfectant at the same treatment concentration. At the tested concentrations used in the experiment, none of the embryos treated with the disinfection solution according to the present invention die, whereas the embryos die under an action of the 84 disinfectant at the concentrations of 20% and 10%. Moreover, the lethal concentration 50 (LC50) of the disinfection solution according to the present invention is between 5% and 10% for the juvenile zebrafishes, while the LD50 of the 84 disinfectant for the juvenile zebrafish is less than 1%. This result demonstrates that the acute toxicity of the novel disinfection solution according to the present invention for the zebrafish is significantly lower than that of the 84 disinfectant.

### II. Behavioral Experiment on Zebrafishes

(1) The disinfection solution and an 84 disinfectant were diluted with an OD solution to make final concentrations reach 0.5%, 0.25%, 0.125%, 0.6125%, and 0.30625% respectively (calculation of volumetric ratios based on concentrations).

(2) 20 zebrafish embryos and 20 juvenile zebrafishes were added to the OD solution containing the disinfection solution at different concentrations, followed by recording the number of tail wagging per minute of the embryos at 24 h and the juvenile zebrafishes' heartbeats per minute at 48 h respectively to evaluate whether the states of the zebrafishes are affected by the disinfection solution. All data was recorded in the form of video shooting. (No positive control group of the 84 disinfectant was set up due to the fact that the zebrafishes could not survive for a long time in 0.5% of 84 disinfectant.)

(3) The videos taken were processed using the ImageJ software to obtain the data of the number of tail wagging per minute of the zebrafish embryos and heartbeats per minute of the juvenile zebrafishes.

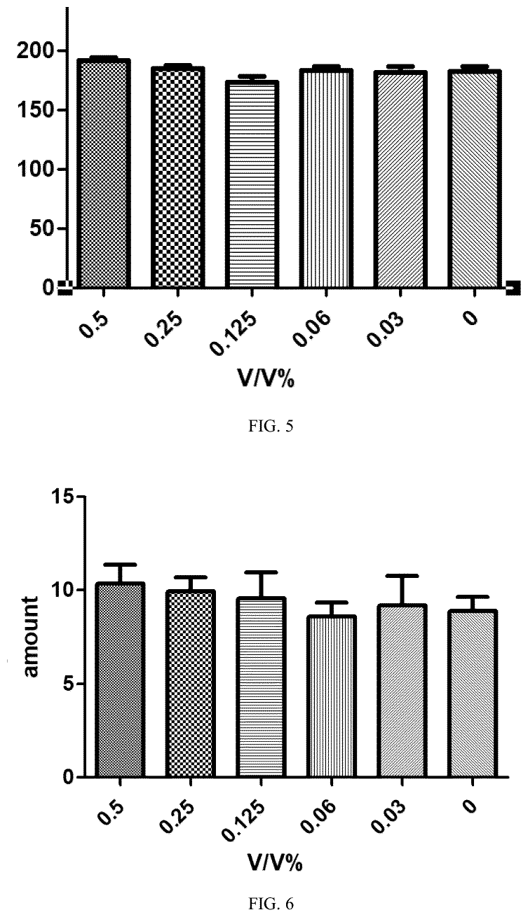

The relevant experiment results are shown in FIG. 5 and FIG. 6: According to the experiment results, it can be seen that there is no significant difference in the basic behavior of zebrafishes in the experimental group treated with the novel disinfection solution according to the present invention and in the blank control group. We can consider that there is no impact on the growth of zebrafishes under the experimental concentrations, which demonstrates that the novel disinfection solution according to the present invention has a relatively low level of ecotoxicity.

### Effect Verification V: In Vivo Toxicity Evaluation Experiment on Disinfection Solution Prepared in Example 1.

This experiment relates to the in vivo toxicity of the disinfection solution based on the citric acid and the natural naphthoquinone compound in Example 1. The toxicity in Balb/C mice is tested using a disinfection solution feeding method.

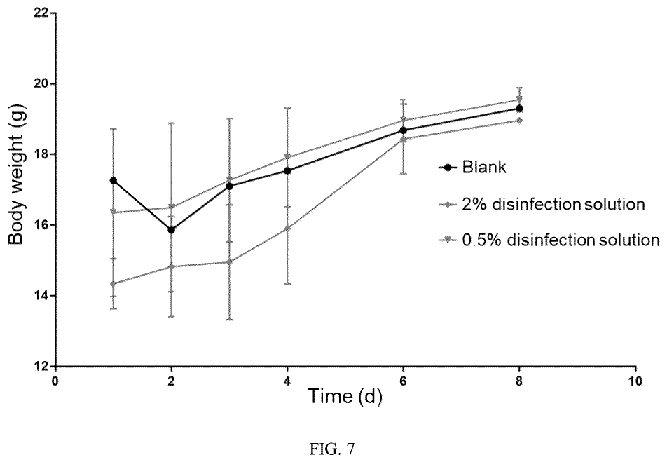

We continuously measured the changes in body weight of the mice within seven days, with the records shown in FIG. 7.

From the experiment results, we observe that, under the feeding conditions of the disinfection solution according to the present invention at the effective bactericidal concentrations, the changes in body weight of the mice within seven days are basically the same as those of the blank control group, which shows that the novel disinfection solution according to the present invention has a low oral toxicity and is safer.

In conclusion, the method for preparing the disinfection solution based on the citric acid and the natural naphthoquinone compound according to the present invention is simple, and the raw materials thereof are easily available. According to the activity assay results, the disinfection solution features high disinfection and sterilization efficacy, low irritation, low corrosiveness, and stable quality, and is free from peculiar smell and environmental toxicity in disinfection residues. The disinfectant is suitable for cold chain disinfection, disinfection of surfaces of vegetable and food, and protection against germ warfare. The application places of the disinfectant include the public places, such as hospitals at all levels, shopping malls, theaters, Internet cafes, restaurants, administrative organizations, enterprises, public transportation tools, and the like, and home.

The specific examples of the present invention are described in the foregoing paragraphs. It should be appreciated that the present invention is not limited to the specific examples described above, and those skilled in the art may make various deformations or modifications within the scope of the claims, which do not affect the substantive content of the present invention.

What is claimed is:

1. A disinfection product based on citric acid and a natural naphthoquinone compound, comprising the following components in parts by weight:
15-25 parts of the citric acid; and
0.01-1 part of the natural naphthoquinone compound.

2. The disinfection product according to claim 1, wherein the natural naphthoquinone compound comprises one or more of juglone, 7-methyljuglone, 7-methyl-5-acetyl juglone, rheum emodin, and alkannin.

3. The disinfection product according to claim 1, further comprising 0.1-1 part of an inorganic salt in parts by weight.

4. The disinfection product according to claim 3, wherein the inorganic salt comprises an alkali metal salt and/or an alkaline-earth metal salt.

5. The disinfection product according to claim 4, wherein the alkali metal salt comprises one or more of sodium chloride, potassium sulphate, and potassium nitrate; and the alkaline-earth metal salt comprises one or more of magnesium chloride, magnesium sulphate, and calcium sulphate.

6. A method of using a disinfection product based on citric acid and a natural naphthoquinone compound in a preparation of a disinfection solution and/or a disinfection gel.

7. A disinfection solution, comprising, per hundred parts, the following components in parts by weight:
15-25 parts of citric acid;
1-5 parts of a cosolvent;
0.01-1 part of a naphthoquinone compound;
0.1-1 part of an inorganic salt; and
a balance of deionized water.

8. A method for preparing the disinfection solution according to claim 7, comprising the following steps:
S1, weighing and adding the citric acid into an appropriate amount of the deionized water to obtain a first mixture, and heating the first mixture to 70-95° C. in a water bath reaction kettle to fully dissolve the citric acid in a preparation process to prepare an aqueous citric acid solution;
S2, weighing and adding the inorganic salt into the aqueous citric acid solution prepared in the step S1 to obtain a second mixture, and stirring the second mixture at a stirring speed of 60-100 r/min at 70-95° C. for 25-35 minutes to obtain a resulting solution; and
S3, cooling the resulting solution obtained in the step S2 to room temperature, followed by adding a mixed solution to obtain a third mixture, and stirring the third mixture evenly to obtain the disinfection solution, wherein the mixed solution is formed by dissolving the naphthoquinone compound into the cosolvent, and a mass fraction of the naphthoquinone compound in the disinfection solution is 0.01-1%.

9. A disinfection gel, comprising, per hundred parts, the following components in parts by weight:
15-25 parts of citric acid;
0.01-0.1 parts of a naphthoquinone compound;
0.1-1 part of an inorganic salt;
1-5 parts of a cosolvent;
1-3 parts of a thickener; and
a balance of deionized water.

10. A method for preparing the disinfection gel according to claim 9, comprising the following steps:
A1, weighing and adding the citric acid into an appropriate amount of the deionized water to obtain a first mixture, and heating the first mixture to 70-95° C. in a water bath reaction kettle to fully dissolve the citric acid in a preparation process to obtain an aqueous citric acid solution;
A2, weighing and adding the inorganic salt into the aqueous citric acid solution obtained in the step A1 to obtain a second mixture, and stirring the second mixture at a stirring speed of 60-100 r/min at 70-95° C. for 25-35 minutes to obtain a resulting solution; and
A3, cooling the resulting solution obtained in the step A2 to room temperature, followed by adding a mixed solution to obtain a third mixture, stirring the third mixture evenly to obtain a disinfection solution, and adding the thickener to the disinfection solution for mixing and thickening to obtain the disinfection gel, wherein the mixed solution is formed by dissolving the naphthoquinone compound into the cosolvent, and a mass fraction of the naphthoquinone compound in the disinfection solution is 0.01-1%.
